(19) United States
(12) Patent Application Publication
(10) Pub. No.: US 2024/0316094 A1
(43) Pub. Date: Sep. 26, 2024

(54) AEROSOL AND TOPICAL ADMINISTRATION OF A FORMULATION CONTAINING CYCLODEXTRIN, QUERCETIN AND ZINC, IN COMBINATION OR SEPARATELY, TO MITIGATE INFECTION BY ENVELOPED VIRUSES

(71) Applicant: Porosome Therapeutics, Inc., Boston, MA (US)

(72) Inventor: Bhanu Pratap Jena, Bloomfield Hills, MI (US)

(21) Appl. No.: 18/680,557

(22) Filed: May 31, 2024

Related U.S. Application Data
(63) Continuation of application No. 17/472,604, filed on Sep. 11, 2021.
Application No. 17/472,604 is a continuation-in-part of application No. 17/207,250, filed on Mar. 19, 2021.
(60) Provisional application No. 63/029,458, filed on May 23, 2020.
(60) Provisional application No. 63/019,312, filed on May 2, 2020.

(51) Int. Cl. A61K 31/724; A61K 9/00; A61K 9/12; A61K 31/352; A61K 33/30; A61K 47/02; A61K 47/18

(52) U.S. Cl. CPC A61K 31/724; A61K 9/008; A61K 9/12; A61K 31/352; A61K 33/30; A61K 47/02; A61K 47/186

(57) ABSTRACT

The invention provides the use of a formulation containing cyclodextrin, quercetin and zinc, at appropriate concentrations to mitigate infections by enveloped viruses like SARS-CoV-2, influenza and HIV/AIDS, when administered via pulmonary and dermal route. While the different forms of cyclodextrin prevent the entry of coated virus into host cells by extracting and sequestering cholesterol molecules at the virus coat and at the host cell plasma membrane, the natural plant-based ionophore quercetin in the formulation, enables cellular entry of zinc, inhibiting viral replication by altering polymerase activity in the host cell. Hence cyclodextrin, and quercetin plus zinc, either in combination or in tandem order of administration, serves both as prophylactic and therapeutic.

### CROSS-REFERENCE TO RELATED APPLICATIONS

The present application is a continuation of U.S. patent application Ser. No. 17/472,604 filed on Sep. 11, 2021, which is a continuation-in-part application of U.S. patent application Ser. No. 17/207,250, filed on Mar. 19, 2021, which claims the benefit of U.S. Provisional Patent Application Ser. Nos. 63/029,458, filed May 23, 2020, and 63/019,312 filed May 2, 2020, and further claims the benefit of U.S. Provisional Patent Application Ser. No. 63/235,772, filed on Aug. 22, 2021, each of which is hereby incorporated by reference herein in their entireties.

### FIELD OF THE INVENTION

The present application generally relates to medicine and, more particularly, to an inhalation and topical use of a formulation containing cyclodextrin, quercetin, and zinc to mitigate infection by enveloped viruses such as HIV and SARS-cov-2.

### DISCUSSION OF ART

Viruses enter hosts via the epithelium. The cell plasma membrane of skin and lung epithelia is the first line of defense and when breached, serves as the portal for viral entry into hosts. Studies in the past two decades report the various cell membrane binding and entry mechanisms utilized by viruses to infect. Irrespective of the different mechanisms involved in viral entry into host cells, the initiating critical process is binding of the virus to the cell plasma membrane. Without binding of virus to the cell plasma membrane, there would be no viral entry into the host.

A large number of studies have established that binding of viruses to the cell plasma membrane is subjected to the presence of docking sites or receptors and their regulation by membrane lipid composition and distribution such as the establishment of domains called rafts. Our recent study involving cellular membrane biogenesis demonstrates that changes in composition of membrane cholesterol impacts both the chemistry and distribution of plasma membrane proteins and lipids, impacting cell function.

In agreement, recent studies demonstrate that depletion of plasma membrane cholesterol in host cells using M-βCD, significantly reduces entry of the pseudorabies and vaccinia virus into cells. Similarly, studies demonstrate that HIV infectivity is critically dependent on cholesterol. Cholesterol microdomains, called lipid‘rafts’, have been suggested in the cellular entry or infection of HIV, its assembly, and its release from infected cells. Studies further report that plasma membrane cholesterol is also required for a wide range of both bacterial and yeast infections. Furthermore, high-cholesterol diet impairs pulmonary host defense against gram-negative bacteria and Mycobacterium tuberculosis. Taken together, these results support that CD-mediated depletion of plasma membrane cholesterol in epithelial cells i.e., skin, nasal passage and lung epithelia in humans, using topological application, aerosol spray and nebulization, will mitigate both viral entry and secondary bacterial and yeast infections.

CDs are a family of cyclic oligosaccharides constituted of a macrocyclic ring of glucose subunits joined by a-1,4 glycosidic bonds. CDs are used for improving the water-solubility and bioavailability of a wide range of drugs. The U.S. Food and Drug Administration (FDA) has approved the use of cyclodextrins since 2001. Cyclodextrins were first employed in the food industry in the 1970s, and since they have been used as food additives for carrying food-related lipophiles such as vitamins, aromas and colorants. βCD has also been used as a cholesterol-reducing agent in food of animal origin such as milk and egg. The first pharmaceutical patent related to CDs and pharmaceutical applicability as complexing agents is dated 1953. Currently, cyclodextrins are employed in pharmaceutical products primarily to increase water solubility of poorly soluble drug formulations and to enhance drug bioavailability. Pharmaceutical products containing CDs comprise nasal spray, oral solutions, solid dosage forms, ocular and dermal formulations, suppositories, and parenteral solutions.

Currently, more than 40 pharmaceutical products containing CDs are available in the market worldwide, and the vast majority of them utilize βCD and its derivatives having higher water solubility such as HPβCD, MβCD, and SBEβCD. Most of the βCD are also approved by the European Medical Agency for all human administration pathways. CDs are used for example in tablets, aqueous parenteral solutions, nasal sprays and eye drop solutions.

Examples of the use of cyclodextrins in medicines on the European market are β-CD in cetirizine tablets and cisapride suppositories, y-CD in minoxidil solution, and examples of the use of R-cyclodextrin derivatives are SBE-β-CD in the intravenous antimycotic voriconazole, HP-β-CD in the antifungal itraconazole, intravenous and oral solutions, and RM-β-CD in a nasal spray for hormone replacement therapy by 17β-estradiol. In Germany and Japan there are infusion products on the market, containing alprostadil (prostaglandin E1, PGE1) with α-CD. Cyclodextrins are currently not included in the European Commission Guideline on excipients in the label and package leaflet of medicinal products for human use. CDs in combination with quercetin and zinc, or used separately in tandem order (CDs followed by Quercetin+Zn), either via pulmonary or dermal route, have never been used as an anti-viral drug until now, as presented here in this application.

### BRIEF DESCRIPTION OF THE INVENTION

Embodiments of the invention provide the use of a formulation containing cyclodextrin, quercetin and zinc, at appropriate concentrations to mitigate infections by enveloped viruses like SARS-CoV-2, influenza and HIV/AIDS. While the different forms of cyclodextrin prevent the entry of coated virus into host cells by extracting and sequestering cholesterol molecules at the virus coat and at the host cell plasma membrane, the natural plant-based ionophore quercetin in the formulation, enables cellular entry of zinc, inhibiting viral replication by altering polymerase activity in the host cell.

Using the non-toxic U.S. Food and Drug Administration (FDA)-approved excipient cyclodextrin as a drug in phosphate buffered saline solutions, will allow the extraction of cholesterol molecules from enveloped virus membranes and the host cell membrane, altering their respective lipid and protein composition and distribution, preventing virus entry into host cells. Using quercetin, a naturally occurring plant-based over the counter zinc ionophore, will enable the cellular entry of zinc to protect host cells against the virus by inhibiting RNA binding, RNA synthesis, viral polyprotein cleavage, viral replication, and viral protease enzyme inactivation, among others. Administration of cyclodextrin alone followed by quercetin and zinc administration or the two combined, in a water based soluble formulation, prevents both viral entry and replication in host cells.

Aerosol spray and nebulization of the combined cyclodextrin, quercetin and zinc, or their tandem administration as an aqueous phosphate buffered saline pH 7.5 solution containing 0.01% Benzalkonium chloride as preservative, will be used to protect the airways including lungs from all coat virus infections. Similarly, topical application of the combined cyclodextrin, quercetin and zinc in aqueous phosphate buffered saline pH 7.5 solution containing 0.01% Benzalkonium chloride as preservative, will be used to protect body surface (skin) from all coated virus infection. Additionally, application to both sides of cellulose masks of the combined cyclodextrin, quercetin and zinc aqueous phosphate buffered saline pH 7.5 solution containing 0.01% Benzalkonium chloride as preservative, will further protect the airways including lungs from all coated virus infection. In such medicated masks, any airborne droplets containing the virus will be neutralized on contact with the medicated mask. This is the first direct use of cyclodextrin, quercetin and zinc as an anti-viral, anti-bactericidal and anti-fungal drug, either in combination or used separately in tandem order (CDs followed by quercetin+Zn) via pulmonary or dermal route.

### DETAILED DESCRIPTION OF THE INVENTION

In this study we report that cells exposed to an increasing concentration of methyl beta cyclodextrin (M-βCD) to deplete cholesterol from the cell plasma membrane demonstrate loss in the uptake of phosphatidyl serine by the cell plasma membrane, while the uptake of phosphatidylethanolamine remain unchanged. Similarly, the loss of cholesterol from the cell plasma membrane resulted in the depletion of membrane fusion proteins such as syntaxin and SNAP25 from the plasma membrane suggesting altered membrane fusogenicity. Therefore, changes to the chemistry of the epithelial cell plasma membrane via depletion of sterols/cholesterol by cyclodextrins (CDs), could dictate both the binding of the virus at the cell plasma membrane, and influence both the efficacy and potency of its entry into the host cell.

Formulation is adapted for both surface use and introduction to a subject by dispersal, utilizing any known method of measured nasal sprays or inhalers, wet wipes and medicated masks. The formulation is introduced in form of an aqueous phosphate buffered saline pH 7.5 solution, containing 0.01% Benzalkonium chloride as preservative and different concentrations of the active ingredients: 1-5% cyclodextrin; the flavonoid quercetin, a naturally occurring zinc ionophore at a concentration of 8 ug/ml; and 1 mg/ml zinc chloride. Depending on requirement, pH 2.5 citrate buffered aqueous will also be used, where the low pH serves as a preservative and a solvent for cyclodextrin. The active ingredient cyclodextrin (CD) is currently being used as an excipient in pharmaceutical products including in nasal sprays. The U.S. Food and Drug Administration (FDA) has approved the use of CDs since 2001. CDs were first employed in the food industry in the 1970s, and since they have been used as food additives for carrying food-related lipophiles such as vitamins, aromas and colorants. The first pharmaceutical patent related to CDs and pharmaceutical applicability, was made in 1953 to serve as a complexing agent. Pharmaceutical products containing CDs comprise nasal spray, oral solutions, solid dosage forms, ocular and dermal formulations, suppositories, and parenteral solutions. Currently, more than 40 pharmaceutical products containing CDs are available in the market worldwide, and the vast majority of them utilize βCD and its derivatives having higher water solubility such as HPβCD, M-βCD, and SBEβCD. Most of the βCDs are also approved by the European Medical Agency for all human administration pathways.

Zinc is an essential trace element supporting growth, development and immune health. Zinc is also known to protect against viruses by inhibiting RNA binding, RNA synthesis, viral polyprotein cleavage, viral replication, and viral protease enzyme inactivation. Zinc, however, needs to enter the host cell to protect against the virus. Quercetin, a naturally occurring plant-based over the counter zinc ionophore, will enable the cellular entry of zinc to protect host cells against the virus. Furthermore, quercetin has shown therapeutic effects against influenza virus. Additionally, in silica modelling of the interactions between the SARS-CoV-2 Viral Spike Protein and the epithelial cell Angiotensin Converting Enzyme-2 (ACE2) protein, has identified quercetin from a database of 8,000 small molecule candidates of known drugs, metabolites, and natural products, as one of the top 5 most potent compounds for binding to the interface site, and disrupt initiation of viral infection.

Benzalkonium chloride, widely used as a preservative in nasal sprays and nebulization, has been reported to cause sino-nasal mucosal injury, nasal squamous metaplasia, ciliary dysmotility, genotoxicity, and other adverse effects (45-47}. Data also suggests the toxic effects of phenylcarbinol, another commonly used preservative (48-50). Despite this evidence, these preservatives continue to be used at higher concentrations even in over-the-counter preparations. Acidification (pH 2.5) of nasal, inhalable, and topical ophthalmic preparations have been demonstrated to maintain sterility without the need for preservatives. This approach of lowering the pH of the formulated CD and quercetin solutions to be used in wipes and aerosol sprays, will preclude the use of harmful preservatives at higher concentrations, without compromising the sterility of the formulation. Therefore, either low concentration of benzalkonium chloride and or low pH formulations will be prepared for use.

Natural CDs such as αCD, βCD, and γCD are hydrophilic in aqueous solutions; however, they tend to self-assemble and form complexes. To overcome this limitation, soluble βCD derivatives such as 2-hydroxypropyl-βCD (HPβCD) and sulfobutylether βCD sodium salt (SBEβCD), are preferred for use in aqueous pharmaceutical formulations. Studies report that inorganic acids such as phosphoric and citric acid induce CD solubilization.

Therefore, embodiments of the present CD formulation will utilize FDA approved concentrations of CDs, quercetin and zinc in buffered solutions to retain both high solubility and sterility. Mode of administration will be through aerosol spray and nebulization, and topical application on body surface using a water-based solution adsorbed to paper, cellulose or fabric. The topical application on body surfaces will include the face and neck, to mitigate envelop virus (such as SARS-CoV-2, influenza and HIV), bacteria and fungus infections.

As a proof, recent clinical studies separately using CDs and quercetin on humans, some using randomized, controlled and open label clinical trial, show 98%-100% effective in providing protection from SARS-CoV-2 infection as determined using RT-qPCR. These studies show that 98% of SARS-CoV-2 infected patients were cleared of the virus in just two weeks of receiving the quercetin treatment.

What is claimed is:

1. An aqueous composition comprising:
a cyclodextrin;
a zinc ionophore;
a zinc-containing compound; and,
a preservative.

2. The aqueous composition of claim 1 wherein the cyclodextrin is at least 1 selected from the group of: hydroxypropyl-β-cyclodextrin, M-βCD, and SBEβCD.

3. The aqueous composition of claim 1 wherein the cyclodextrin is at 1-5% of the composition.

4. The aqueous composition of claim 1 wherein the zinc ionophore is quercetin.

5. The aqueous composition of claim 1 wherein the preservative is benzalkonium.

6. The aqueous composition of claim 5 wherein the benzalkonium is at 0.1% of the composition.

7. The aqueous composition of claim 1 wherein the zinc-containing compound is zinc chloride.

8. The aqueous composition of claim 1 further comprising a buffer.

9. The aqueous composition of claim 8 wherein the buffer is a phosphate buffer.

10. A method of reducing enveloped virus number comprising:
administering to a subject in need thereof the aqueous composition of claim 1.
